(19) United States
(12) Patent Application Publication
(10) Pub. No.: US 2001/0032345 A1
(43) Pub. Date: Oct. 18, 2001

(54) Apple variety

(76) Inventor: Alois Braun, Girlan (IT)

Correspondence Address: Horst M. Kasper, 13 Forest Drive, Warren, NJ, 07059, US

(21) Appl. No.: 09/829,183

(22) Filed: Apr. 9, 2001

(30) Foreign Application Priority Data
Jan. 13, 2000 (ZA) PT2955
Oct. 27, 2000 (NL) APP0205

(51) Int. Cl. A01H 5/00

(52) U.S. Cl. PLT/168

(57) ABSTRACT

The present invention relates to a variety of an apple tree obtained by branch mutation and by selection of the variety Fuji. The present invention variety exhibits an appropriate distinguishability, homogeneity, uniformity, and stability. The vigor of the tree is strong. The tree type is ramified. The habit of the apple tree is drooping. The pubescence on an upper half of a shoot is weak. The thickness as measured by the diameter at the center is medium for a dormant one year old shoot. The number of lenticells is many for one year old dormant shoot. The color of an unopened flower is light pink in a balloon stage. The surface of the fruit is colored red with a large number of small yellowish dots. Frequently the dots are disposed on curved lines with each line counting from about 4 to 8 dots.

### BACKGROUND OF THE INVENTION

1. Field of the Invention

The present invention relates to a novel apple tree.

2. Brief Description of the Background of the Invention Including Prior Art

A known variety of an apple plant is malus domesticus. A variety of malus domesticus is called Fuji.

### BRIEF SUMMARY OF THE INVENTION

A vegetatively propagated for variety of “Malus Domesticus” is designated as “BRAK”. To variety has been bred by a branch mutation of the variety Fuji by selection such that the variety “BRAK” exhibits necessary distinguishability, homogeneity, uniformity, and stability.

### BRIEF DESCRIPTION OF THE DRAWING

FIG. 1 is a side elevational view of a fruit of the apple tree;

FIG. 2 is a side elevational view of another fruit of the apple tree;

FIG. 3 is a perspective view of a hanging fruit of the apple tree showing part of the bottom of the fruit;

FIG. 4 is a side elevational view of three fruits hanging from an apple tree;

FIG. 5 is a view of an apple tree carrying a plurality of fruits.

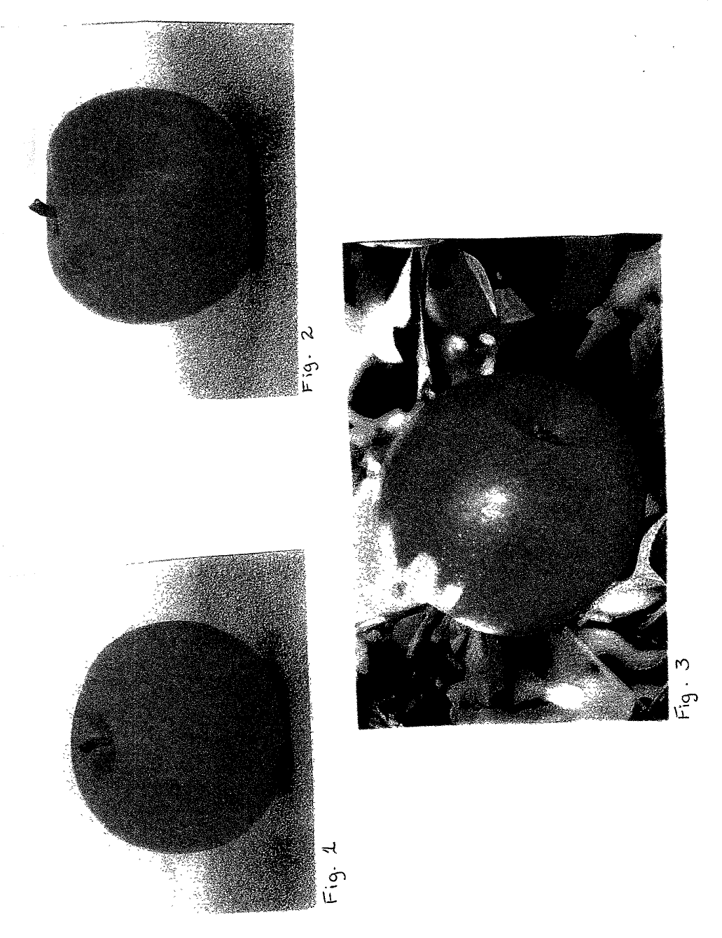

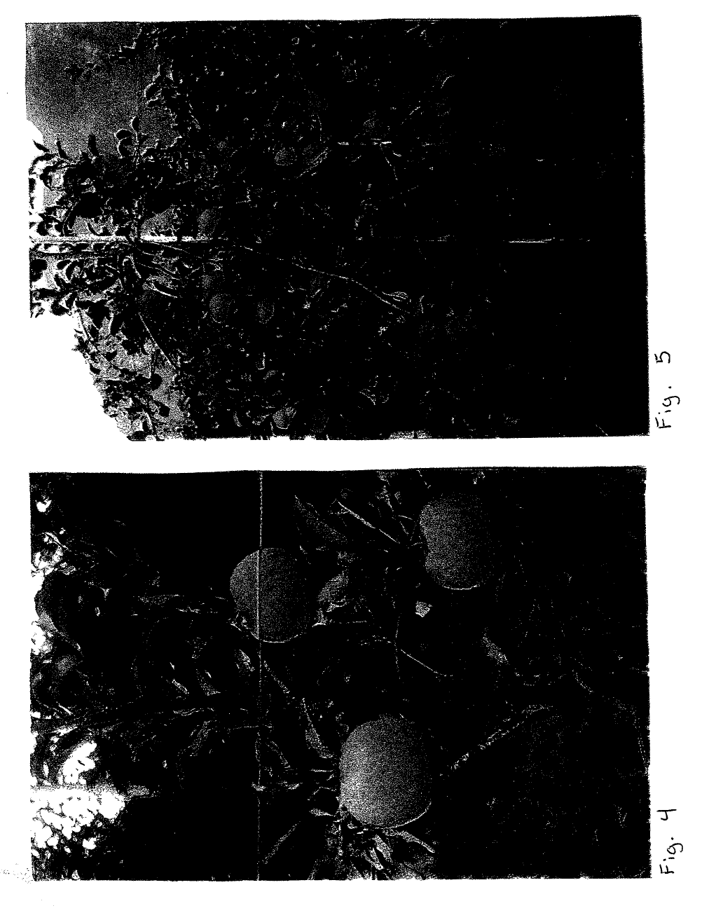

### DETAILED BOTANICAL DESCRIPTION

The present invention relates to a variety of an apple tree. A branch mutation of the variety Fuji has been performed by selection such that the present invention variety exhibits an appropriate distinguishability, homogeneity, uniformity, and stability.

The vigor of the tree is strong. The tree type is ramified. The habit of the apple tree is drooping. The pubescence on an upper half of a shoot is weak. The thickness as measured by the diameter at the center is medium for a dormant one year old shoot. The length of the internode is medium for a dormant one year old shoot. The number of lenticells is many for one year old dormant shoot. The color of an unopened flower is light pink in a balloon stage. The size of the flower as measured by the diameter of the flower with petals pressed into a horizontal position is medium. The relative position of the margin of the petals is touching. The attitude of the leaf relative to the shoot is outwards. The length of the leaf blade is medium. The width of the leaf blade is medium. The ratio length to width of the leaf blade is medium. The incisions of the margins of the leaf blade are serrate. The length of the petiole is medium. The size of the fruit is large. The ratio of height to width of the fruit is medium. The position of the maximum width of the fruit is toward the stalk. The shape of the fruit is globose. The prominence of ribbing of the fruit is absent or very weak. The crowning at the calyx end is absent or very-weak. The aperture of the eye of the fruit is partly open. The size of the eye of the fruit is medium. The visual length of the sepal of the fruit is medium. The depth of the eye basin is deep. The width of the eye basin is broad. The thickness of the stalk of fruit is medium. The length of the stalk of the fruit is long. The depth of the stalk cavity of the fruit is medium. The width of the stalk cavity of the fruit is broad. The relief of the surface of the fruit is smooth. The bloom of the skin of the fruit is absent. The greasiness of the skin of the fruit is absent. The color of the ground color of the skin of the fruit if visible is green yellow. The color of the over color of the skin of the fruit is red. The intensity of the color of the over color of the skin of the fruit is medium. The amount of over color of the skin of the fruit is very high. The pattern of over color of the skin of the fruit is striped. The amount of russet around are the eye basin of the fruit is absent or very low. The amount of russet on the cheeks of the fruit is absent or very low. The amount of russet around the stalk cavity of the fruit is low. The size of the lenticells of the fruit is medium. The firmness of the flesh of the fruit as measured with a penetrometer is firm. The color of the flesh of the fruit is yellowish. The aperture of the locules for a median through locules of the fruit in cross-section is partly open.

The time of the beginning of flowering as measured by 10 percent open flowers is medium. The time of fruit ripening for eating is late.

The surface of the fruit is colored red with a large number of small yellowish dots. Frequently the dots are disposed on curved lines with each line counting from about 4 to 8 dots. The diameter of the dots is generally less then 0.5 mm and nearly always less then a millimeter. The total number of dots is estimated to be between from about five hundred to five thousand for a single fruit.

I claim:

1. A variety of an apple tree derived by a branch mutation of the variety Fuji, wherein the apple tree exhibits a strong vigor, wherein the tree type is ramified, wherein the habit of the apple tree is drooping, wherein the pubescence on an upper half of a shoot is weak, wherein the thickness as measured by the diameter at the center is medium for a dormant one year old shoot, wherein the length of the internode is medium for a dormant one year old shoot, wherein the number of lenticells is many for one year old dormant shoot, wherein the color of an unopened flower is light pink in a balloon stage, wherein the size of the flower as measured by the diameter of the flower with petals pressed into a horizontal position is medium, wherein the relative position of the margin of the petals is touching, wherein the attitude of the leaf relative to the shoot is outwards, wherein the length of the leaf blade is medium, wherein the width of the leaf blade is medium, wherein the ratio length to width of the leaf blade is medium, wherein the incisions of the margins of the leaf blade are serrate, wherein the length of the petiole is medium. The size of the fruit is large, wherein the ratio of height to width of the fruit is medium, wherein the position of the maximum width of the fruit is toward the stalk, wherein the shape of the fruit is globose, wherein the prominence of ribbing of the fruit is absent or very weak, wherein the crowning at the calyx end is absent or very weak, wherein the aperture of the eye of the fruit is partly open, wherein the size of the eye of the fruit is medium, wherein the visual length of the sepal of the fruit is medium, wherein the depth of the eye basin is deep, wherein the width of the eye basin is broad, wherein the thickness of the stalk of fruit is medium, wherein the length of the stalk of the fruit is long, wherein the depth of the stalk cavity of the fruit is medium, wherein the width of the stalk cavity of the fruit is broad, wherein the relief of the surface of the fruit is smooth, wherein bloom of the skin of the fruit is absent, wherein greasiness of the skin of the fruit is absent, wherein the color of the ground color of the skin of the fruit if visible is green yellow, wherein the color of the over color of the skin of the fruit is red, wherein the intensity of the color of the over color of the skin of the fruit is medium, wherein the amount of over color of the skin of the fruit is very high, wherein the pattern of over color of the skin of the fruit is striped, wherein the amount of russet around are the eye basin of the fruit is absent or very low, wherein the amount of russet on the cheeks of the fruit is absent or very low, wherein the amount of russet around the stalk cavity of the fruit is low, wherein the size of the lenticells of the fruit is medium, wherein the firmness of the flesh of the fruit as measured with a penetrometer is firm, wherein the color of the flesh of the fruit is yellowish, wherein the aperture of the locules for a median through locules of the fruit in cross-section is partly open, wherein the surface of the fruit is colored red with a large number of small yellowish dots, and wherein the dots are frequently disposed on curved lines.
